(19) United States
(12) Patent Application Publication
(10) Pub. No.: US 2009/0118632 A1
(43) Pub. Date: May 7, 2009

(54) Effort-Independent, Portable, User-Operated Capnograph Devices And Related Methods

(76) Inventor: Julius G. Goepp, Rochester, NY (US)

Correspondence Address: LATHROP & GAGE LC, 4845 PEARL EAST CIRCLE, SUITE 201, BOULDER, CO, 80301, US

(21) Appl. No.: 12/265,574

(22) Filed: Nov. 5, 2008

Related U.S. Application Data
(60) Provisional application No. 60/985,416, filed on Nov. 5, 2007.

(51) Int. Cl. A61B 5/08

(52) U.S. Cl. 600/532

(57) ABSTRACT

An effort-independent, portable, user-operated capnograph device for evaluating a pulmonary status of a user. At least one baseline measurement is stored within a memory of the capnograph device. The identity of a user of the capnograph device is verified to prevent miss-use. Concentrations of carbon dioxide (CO2) exhaled by the user when breathing normally are sensed and stored within the memory. The stored data is compared with the at least one of the baseline measurements to determine the pulmonary status of the user, and the determined pulmonary status is indicated to the user.

### CROSS-REFERENCE TO RELATED APPLICATIONS

This application claims priority to U.S. Provisional Patent Application No. 60/985,416, filed 5 Nov., 2007, which is incorporated herein by reference.

### BACKGROUND

A capnograph is a non-invasive device that continuously measures the concentration of carbon dioxide (hereinafter CO₂ concentration) of exhaled air using an infrared (1R) beam of light. The proportion of the IR beam absorbed by the exhaled air correlates with the number of CO₂ molecules present in that exhaled air. The time-dependent function of a capnograph yields a capnogram. A capnogram is a waveform or a graph that plots the CO₂ concentration (also known as partial pressure of CO₂, and denoted herein as pCO₂) against time and is usually measured in mmHg (millimeter of Mercury). The shape of the time-dependent CO₂ waveform has certain predictable characteristics in health and illness that vary depending on the degree of airway obstruction in patients having obstructive pulmonary disease.

Obstructive pulmonary disease may be chronic, acute, or acute-on-chronic. The most common forms of chronic obstructive pulmonary disease include asthma and emphysema, both of which are subject to acute-on-chronic exacerbations; some patients may have both diseases. Acute exacerbations as previously mentioned, as well as other pulmonary diseases, can be life-threatening. Typically, a capnograph is used by a skilled medical provider to detect changes in the CO₂ concentration and to determine the severity of exacerbations as indicated by a rising absolute level of CO₂.

Common devices used to monitor the severity of asthma and other obstructive pulmonary diseases and to detect or predict exacerbations include peak flow meters and flow-volume pulmonary function test devices.

Peak flow meters give results that are effort dependent; results are reliable only if a cooperative patient uses well-timed and appropriate muscular effort while exhaling through the meter. Similarly, the most important measurements to quantify asthma and other obstructive pulmonary diseases are usually made with flow-volume pulmonary function test devices as described above. Typical tests using such devices are the Forced Expiratory Volume in 1 second (FEV1) and the ratio of FEV1 to Forced Vital Capacity (FVC). The FEV1 and FVC measurements are effort-dependent because they are accurate only if a cooperative patient inhales as deeply as possible, then exhales as quickly and completely as possible through the meter.

Young children, elderly, injured, anesthetized, sore, and ill patients are often not fully able to cooperate with peak flow and other standard pulmonary function measurements.

FIG. 1A illustrates one exemplary crenellated shaped CO₂ waveform of a healthy person. Segment 101A represents a period immediately prior to expiration, during which CO₂ levels are undetectable. The initial expiration portion of the waveform displays a rapid ascent in pCO₂ as demonstrated by segment 101B on the waveform. As expiration continues, the rate of rise in CO₂ levels slow (segment 101C on the waveform) and reach a constant rate (segment 101D on the waveform). The constant CO₂ expiration ends at 101E where a normal end-expiratory CO₂ value is about 40 mmHg. The segment 101F on the waveform illustrates the rapid decline in detected CO₂ as the patient enters into the next inspiration.

FIG. 1B shows an exemplary embodiment of a CO₂ waveform of a person with pulmonary obstructive disease. As shown in FIG. 1B, in the pulmonary obstructive waveform, the capnogram loses its rectangular crenellated shape for a shark-fin appearance. Segment 101G represents a period immediately prior to expiration, during which CO₂ levels are undetectable. The initial expiration portion of the waveform displays an ascent in pCO₂ (segment 101H), without exhibiting a period of time with a constant expiration of pCO₂. The segment 101J on the waveform illustrates the rapid decline in detected CO₂ as the patient enters into the next inspiration.

Typically, a capnogram is printed out as a paper chart and is kept in a file history. A health care provider is able to determine the pulmonary status of an individual by evaluating the deformation of the shape and slopes of the CO₂ waveform of the capnogram and is able to correlate the shape of the curve indices to the degree of the pulmonary obstruction. However, this identification process is performed retrospectively (i.e., not in real time), requires extensive training and is overall cumbersome. Furthermore, decisions based upon the capnograph information are made by a skilled clinician, not by the patient or the immediate caregivers. In actual use, such detailed analyses of capnogram waveforms are rarely carried out at all, resulting in the loss of potentially-useful information.

### SUMMARY

An effort-independent, portable capnograph device is operated by a user, wherein the user is the patient or the user is a person other than the patient. For example, the user may include a trained medical professional or a person without medical training. Hereinafter, the user will be referred to, but that is not to be taken as a limitation of the device.

In one embodiment, an effort-independent, portable, user-operated capnograph device, includes a sensor for sensing concentrations of carbon dioxide (CO₂) exhaled by a user when breathing normally. A memory stores data of the sensed concentrations of CO₂ and one or more baseline measurements. An analyzer compares the stored data with at least one of the baseline measurements to determine the pulmonary status of the user. An output device then indicates the pulmonary status to the user.

In another embodiment, a method evaluates a user's pulmonary status using a portable, user-operated capnograph device. At least one baseline measurement is stored within a memory of the capnograph device. The identity of a user of the capnograph device is verified to prevent miss-use. Concentrations of carbon dioxide (CO₂) exhaled by the user when breathing normally and sensed and stored within the memory. The stored data is compared with at least one of the baseline measurements to determine the pulmonary status of the user, and the determined pulmonary status is indicated to the user.

### BRIEF DESCRIPTION OF THE FIGURES

FIG. 1A shows one exemplary CO₂ waveform of a healthy person.

FIG. 1B shows one exemplary CO₂ waveform of a person with pulmonary obstructive disease.

FIG. 2 shows one exemplary effort-independent, portable, user-operated capnograph device, according to an embodiment.

FIG. 3A illustrates an exemplary housing of the effort-independent, portable, user-operated capnograph device of FIG. 2, according to multiple embodiments.

FIG. 3B illustrates one exemplary bar graph as shown on the display of the effort-independent, portable, user-operated capnograph device of FIG. 2.

FIG. 4 is flow chart illustrating one exemplary process for determining and displaying pulmonary status to a user, according to an embodiment.

FIG. 5A shows exemplary analysis of a CO₂ waveform of a healthy person.

FIG. 5B shows exemplary analysis of a CO₂ waveform of a person with pulmonary obstructive disease.

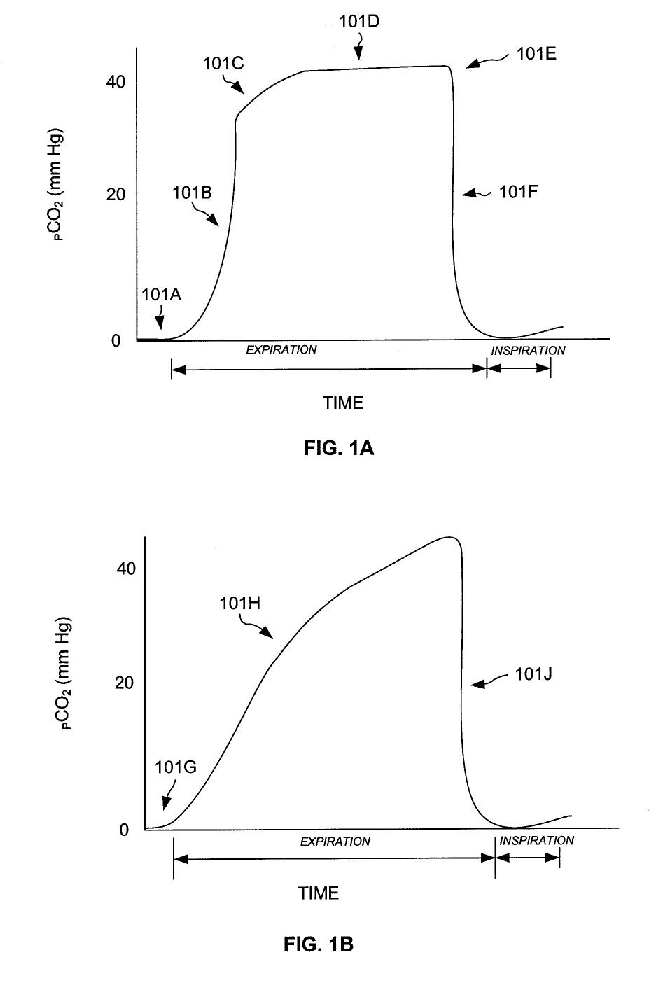

### DETAILED DESCRIPTION OF THE FIGURES

Effort-independent, portable, user-operable capnograph devices and related methods for detecting early changes in pulmonary function are disclosed herein. Effort-independent means that the user simply breathes normally into the mouthpiece or mask, thus the invention allows ease of use for young, sick, injured, and elderly patients. User-operable means operable by people without medical training as well as by a trained medical professional. A “portable” device, for example, enables the user to carry the device at all times. For example, the effort-independent, portable, user-operable capnograph device operates to detect an anomaly of pulmonary function; the device may alert the user, and/or a caregiver, of monitored pulmonary status and allows appropriate action to be taken, such as administering rescue medications, visiting a physician, going to an emergency department, or the like.

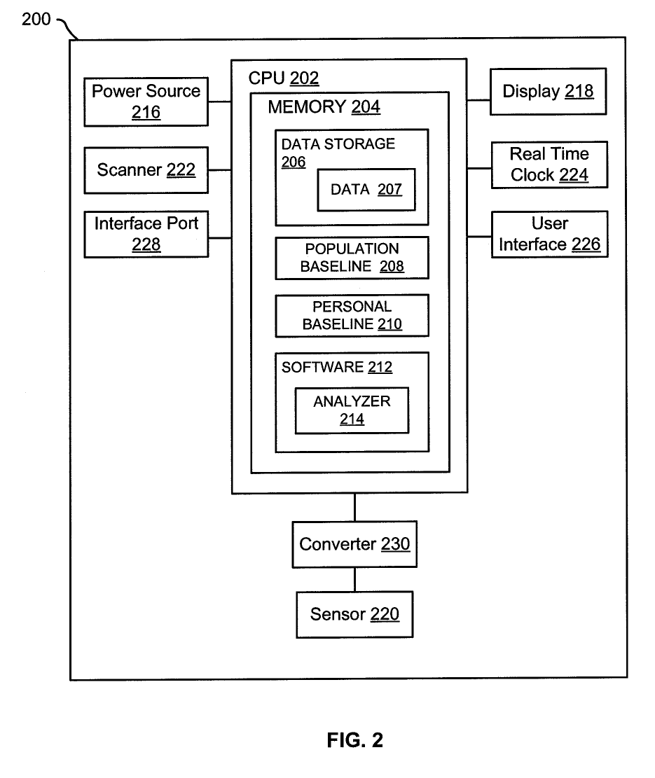

FIG. 2 shows one exemplary portable, user-operated capnograph device 200. Device 200 includes a power source 216, a central processing unit (CPU) 202, a display 218, a sensor 220, a finger print scanner 222, a real-time clock 224, a user interface 226, an interface port 228, and a converter 230. Power source 216 may be a battery, a power adaptor, solar power, and the like. Interface port 228 may include a USB port and/or other serial type interfaces. Device 200 records, analyzes, and alerts the user of pulmonary status of the monitored party.

In an embodiment, device 200 employs CPU 202 to record and analyze a user's pulmonary status for an exhalation session. For example, user interface 226 allows the user to enter user identification data and to interact with CPU 202. User interface 226 may include a keypad or button for entering personal information and/or for selecting information to be displayed on display 218 and/or for setting and clearing alarms. In an embodiment, user interface 226 includes a microphone to receive voice commands from the user, and may include one or more buttons and/or switches that allow the user to select information to be displayed and/or to set an alarm. Real time clock 224, peripheral to CPU 202, supplies date and time stamp of each exhalation session. CPU 202 acts as a controller for device 200.

In an embodiment, device 200 uses fingerprint scanner 222 to identify the user. In one example of operation, fingerprint scanner 222 scans the user's fingerprint and sends the associated fingerprint data to the CPU 202 for comparison against stored fingerprint data of the user. If CPU 202 determines that the received fingerprint data matches the stored fingerprint data, the user's identity is verified. CPU 202 may be a microprocessor that controls device 200. This identity verification prevents miss-use.

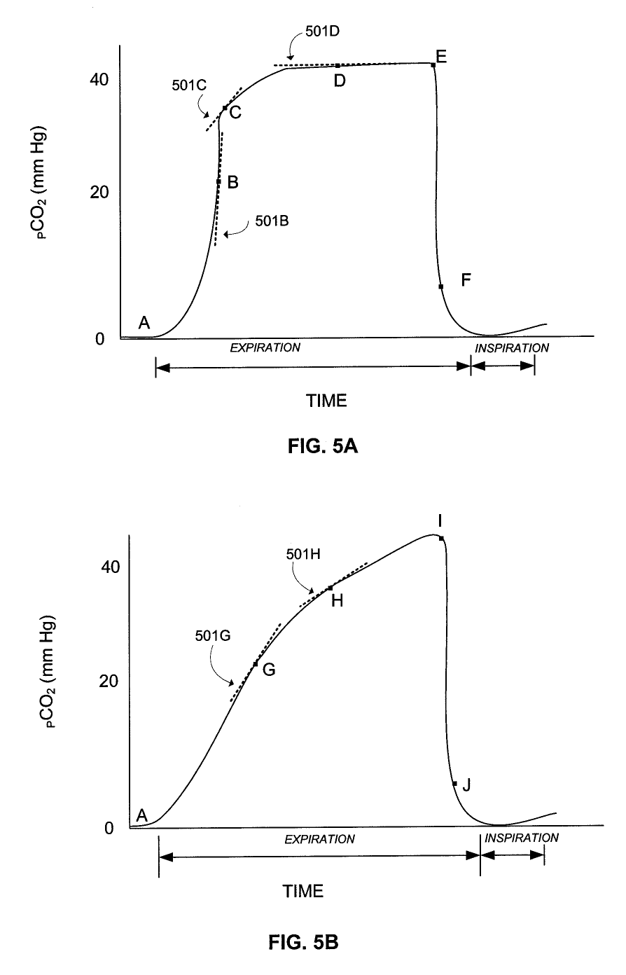

CPU 202 includes a memory 204 having data storage 206, population baseline 208 and/or personal baseline 210, and software 212 that includes an analyzer 214. Once the user's identity is verified, CPU 202 controls sensor 220 to measure the CO₂ concentration of the user's exhaled air during an exhalation session (also known as the exhalation phase). Sensor 220 uses infrared (IR) light to measure the CO₂ concentration of the exhaled air for the duration of the exhalation session. Sensor 220 collects CO₂ concentration content data from successive normal breaths and sends the CO₂ concentration data to converter 230. Converter 230 converts the sensed signal into a format suitable for input to CPU 202 where it is annotated with a date and time stamp received from real time clock 224 and stored within data storage 206 of memory 204 as data 207. Converter 230 represents an electronic signal conditioning circuit, for example. Examples of data 207 are graphically illustrated as waveforms in FIGS. 5A and 5B.

CPU 202 then executes analyzer 214 to perform mathematical waveform analysis of data 207. Analyzer 214 compares the graphical waveform (e.g., the shape or the slope of the waveform) of data 207 with a waveform representative of a population baseline 208. Population baseline 208 is a baseline waveform derived from a series of pulmonary indices of population-based norms. Population baseline 208 may include data representative of expected normal pulmonary function adjusted for age, height and/or weight, and the like, of the normal population. Population baseline 208 is stored within memory 204.

In an embodiment, analyzer 214 compares data 207 with personal baseline 210. Personal baseline 210 includes data representative of the user's personal pulmonary function as measured during previous healthy sessions. In one example, the user instructs CPU 202 to store data 207 as personal baseline 210. Personal baseline 210 may be automatically adjusted based upon the user's developmental stage and/or variation in the user's daily routine, thereby allowing personal baseline 210 to “grow” with the user. For example, a child's weight changes rapidly with normal growth, and the child may have variations in daily routine that are incorporated into personal baseline 210. Establishing a “growing” personal baseline 210 requires the use of device 200 on a regular (e.g., daily) basis.

Daily use of device 200, as is currently recommended for traditional peak flow measurements, enables the user to have an up-to-date personal baseline 210 stored in memory 204, such that subtle changes in the user's pulmonary status may be detected during the course of the day. For example, daily use of device 200 to monitor the user's pulmonary status allows device 200 to continually re-calibrate personal baseline 210 and to establish a trend of the user's pulmonary status. Using an up-to-date personal baseline 210 for analysis provides a more precise evaluation of the user's pulmonary status in comparison to using population baseline 208, because personal baseline 210 adjusts to the user's changes in daily routine and is therefore more attuned to the user. The ability of device 200 to detect subtle changes may also be useful in school, camp and other situations.

In one example of operation, CPU 202 executes software 212, an in particular analyzer 214, to conduct real-time mathematical waveform analysis of data 207 received from converter 230. Analyzer 214 compares, using mathematical waveform analysis, data 207 against recorded population baseline 208 and/or personal baseline 210 and may further determine the trend of the user's pulmonary status. Software 212 may then control CPU 202 to display the determined pulmonary status on display 218 along with specific instruction of appropriate action to take.

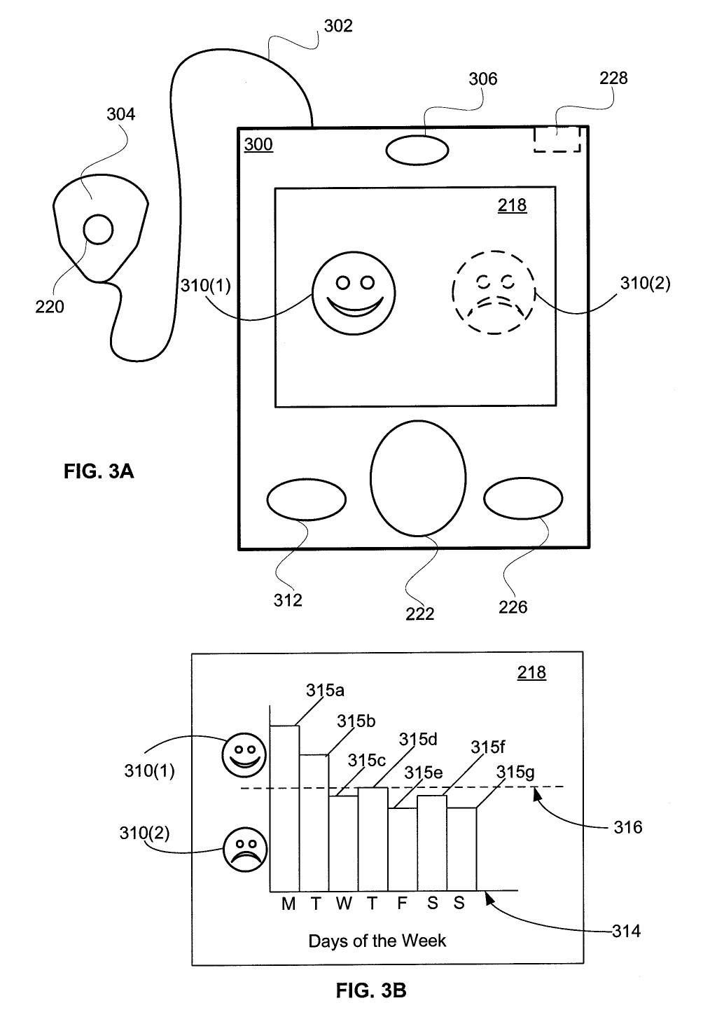

FIG. 3A illustrates an exemplary housing 300 and detection device 304 of device 200, FIG. 2, according to one embodiment. Housing 300 is connected to detection device 304 by a connecting device 302. Housing 300 is shown with a LED 306, interface port 228, display 218, fingerprint scanner 222, user interface 226, and a speaker 312. Speaker 312, display 218 and LED 306 may operate together, independently, or in any combination, to provide user pulmonary status and/or instruction.

Detection device 304 may be a mouth piece and/or a mask to receive exhaled air from the user. Where the user is an infant or is asleep, detection device 304 may represent a side-stream nasal cannula that receives exhaled air. As shown in FIG. 3A, sensor 220 may be included within detection device 304 to measure the CO₂ concentration of the exhaled air. Accordingly, connecting device 302 represents a cable that transmits electrical signals from sensor 220 to converter 230 (not shown) within housing 300. In an alternate embodiment, sensor 220 resides within housing 300 and connecting device 302 is a capillary tube that transports exhaled air to sensor 220.

Display 218 may show the determined pulmonary status and/or instruction as graphical icons, such as a happy face 310(1) or a sad face 310(2). For example, to indicate poor pulmonary status, display 218 shows sad face 310(2) and LED 306 blinks red to attract the user's attention. Simultaneously, speaker 312 may output verbal instructions, such as “call a physician” or “Use rescue inhaler NOW if you have not done so in the past hour.” In one embodiment, the verbal instructions are output using a synthetic voice. In another embodiment, the verbal instructions are output using a recorded message of a parent's voice, thereby appealing more to a child. In an embodiment, device 200 may display messages regarding appropriate corrective measures (i.e., actions) to be taken by the user, where such measures are previously determined by the user and their clinician/physician.

Interface port 228 may be used to transfer stored data, such as trend of the user's pulmonary status, history of user's pulmonary status, to an external device (not shown), such as a computer (e.g., the user's home computer, a physician's office computer, and an emergency department computer) and/or a printer (in the form of a capnogram). Interface port 228 may also represent wireless connectivity, such as Bluetooth, a serial infrared (IR) remote interface, or the like, that is used for transferring data and for connecting with one or more external devices.

FIG. 3B illustrates one exemplary bar graph 314 as shown on display 218, FIG. 2. Bar graph 314 indicates pulmonary status of the user over a selected period of time. The period of time is for example selected from the group comprising: an hour, a day, a week, a month, a year, or is input by the user. In the example of FIG. 3B, bar graph 314 has seven bars 315a-315g that represents a week of recorded pulmonary status for the user. A threshold line 316 separates normal from abnormal pulmonary status ranges for the user. In the example of FIG. 3B, bar 315a crosses threshold line 316 and thus represents pulmonary status within a normal range as indicated by happy face icon 310(1). Bar 315c, on the other hand, remains below threshold line 316 and represents pulmonary status within an abnormal range as indicated by sad face icon 310(2). Bar graph 314 thus shows the user's pulmonary status for the week for assimilation at a glance. This example shows the pulmonary status of the user was normal for two days and abnormal for five days of the week.

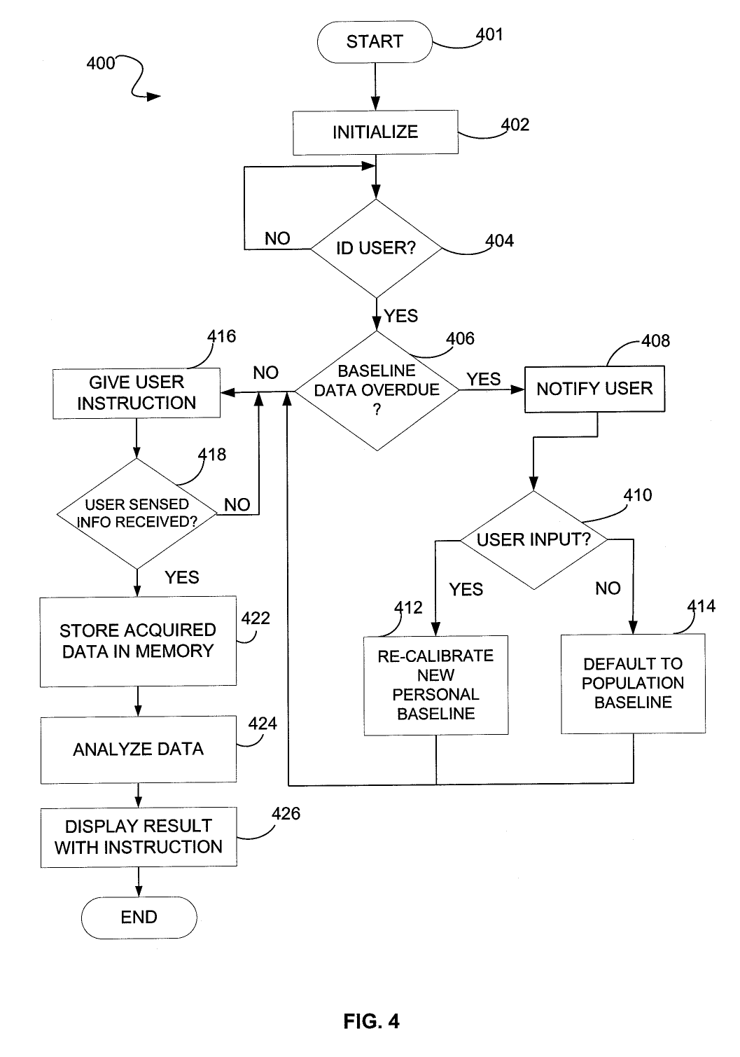

FIG. 4 is flow chart illustrating one exemplary process 400 for determining and displaying pulmonary status to a user. Process 400 is for example implemented within software 212 and executed by CPU 212 of device 200, FIG. 2. Process 400 starts with step 401. In one example of step 401, device 200 is turned on, whereupon display 218 and/or LED 306, FIG. 3A, indicates device activation (e.g., display 218 may show the word “START” and/or LED 306 may flash intermittently. In step 402, process 400 initializes. In one example of step 402, device 200 performs one or more self-checks and calibrations, during which device 200 may show a “READY”/“NOT READY” indication on display 218 and/or LED 306.

Step 404 is a decision. If, in step 404, process 400 identifies the user (e.g., through operation of fingerprint scanner 222, password entry or other conventional identification means), process 400 continues with step 406; otherwise process 400 repeats step 404. Step 406 is also decision step. If, in step 406, process 400 determines that personal baseline 210 measurement of the current session is overdue, process 400 continues with step 408; otherwise, process 400 continues with step 416. In one example of step 406 (determining if a baseline is overdue), software 212 determines that personal baseline 210 data has been stored for a defined period of time (e.g. five to seven days since the baseline measurement was recorded) and continues with step 408.

In another example, the user and/or his/her caregiver set an alarm to alert the user when personal baseline 210 is about to expire. For example, a voice may say “baseline measurement is about to expire, would you like to re-calibrate a new baseline?” If the user does not respond to the voice command via user interface 226, device 200 automatically defaults to using population baseline 208 as a standard for comparison in the next assessment session. Alternatively, the user may be asked (or the device pre-programmed) to use the most recent personal baseline data anyway, and then prompt the user to input new data for use as a personal baseline when an acute exacerbation is resolved. Software 212 may maintain timers (e.g., within CPU 202 and/or real time clock 224) for determining the age of baseline measurement stored in memory 204 and may thereby determine when a new personal baseline 210 measurement is overdue. In one example of normal use, the user may be instructed by device 200 to record personal baseline data every day at a standard time.

In step 408, process 400 notifies the user that personal baseline 210 is overdue via speaker 312, graphical icons 310, or LED 306. Step 410 is a decision. If, in step 410, the user interacts with the device, process 400 continues with step 412; otherwise process 400 continues with step 414. In step 412, process 400 re-calibrates a new personal baseline; the new personal baseline is adjusted to the user's different stages of development such as gain (or loss) of weight or the changes in daily routine, thereby process 400 may “grow” with the individual user. Process 400 then continues with step 416.

In step, 414, device 200 defaults to stored population baseline 208, and provides an indication of the user's status derived from comparison of acquired waveform characteristics in comparison with that data. Step 414 is useful for a distressed user who may have an asthma attack episode and cannot interact with device 200. Process 400 then continues with step 416.

In step 416, process 400 instructs the user. In one example of step 416, software 212, via speaker 312, instructs the user to, for example, “put the mask on and breathe normally”. Normal breathing is all that is required since device 200 is effort-independent; that is, the user does not have to forcefully exhale air as required for FEV1 and peak-flow testing.

Step 418 is a decision. If, in step 418, the user has followed the instructions of step 416 correctly and sufficient CO₂ data has been collected to derive a graphical waveform that is mathematically acceptable (e.g. fits within pre-established parameters for resembling a curve illustrated in FIG. 5), process 400 then continues with step 422; otherwise, process 400 returns to step 416 to give the user instruction to repeat the breathing step; process 400 may suggest remediation on return to step 416 (e.g., “Be sure the mask is properly on your face.”)

In step 422, process 400 stores the acquired data in data storage 206 of memory 204. In one example of step 422, software 212 stores acquired data from sensor 220 (via converter 230) in data storage 206 as data 207. In step 424, process 400 analyzes the characteristics of the data acquired in step 422. In one example of step 424, analyzer 214 mathematically compares at least one characteristic (e.g., slope of a line tangent to a point on the graphical waveform represented by data 207) of data 207 with the corresponding characteristic(s) of at least one of population baseline 208 and/or personal baseline 210. Step 424 also determines if the graphical waveform represented by data 207 falls within or without pre-determined ranges associated with the baseline characteristics.

In step 426, if the characteristics of the graphical waveform represented by data 207 falls within the rep-determined ranges, process 400 displays a positive (good) indication such as a happy face icon 310(1) on display 218, voice information or verbal output from speaker 312. If the characteristics of the graphical waveform represented by data 207 falls outside of the range, then process 400 displays a negative (bad) indication such as a sad face icon 310(2) on display 218, and a warning voice or tone or verbal output from speaker 312. If pre-determined by the user and/or the user's healthcare provider, the display may also include specific instructions such as “use 2 puffs of your rescue inhaler.” If configured for internet access (e.g., through interface port 228 configured as a Bluetooth, WiFi or equivalent interface), an alert may also be sent to the user's parent(s), caregiver, and/or health care provider.

A non-limiting example of data 207 (as analyzed in step 424 of process 400) is shown in FIG. 5A. In particular, FIG. 5A illustrates one exemplary crenellated shaped CO₂ waveform of a healthy person. Segment A, represents a period immediately prior to expiration, during which CO₂ levels are undetectable. The initial expiration portion of the waveform displays a rapid ascent in pCO₂. For example, a tangent line 501B of the waveform at point B shows an ascending slope (i.e., a slope approaching one) as CO₂ levels in expired air rise rapidly. As expiration continues, the slope of a tangent line 501C of the waveform at point C decreases, indicating a slowing of the rate of rise of CO₂ levels. A tangent line 501D of the waveform at point D has a slope of substantially zero as the level of expired CO₂ remains nearly constant. The constant CO₂ expiration ends at point E where a normal end-expiratory CO₂ value is about 40 mmHg. The segment between points E and F on the waveform illustrate the rapid decline in detected CO₂ as the patient enters into the next inspiration.

Another non-limiting example of data 207 (as analyzed in step 424 of process 400) is shown in FIG. 5B. In particular, FIG. 5B illustrates one exemplary CO₂ waveform (capnogram) of a person with pulmonary obstructive disease. As shown in FIG. 5B, the waveform loses its rectangular crenellated shape for a shark-fin appearance. Although, the initial expiration portion of the waveform displays an ascent in pCO₂, a tangent line 501G of the waveform at point G is less steep than the corresponding tangent line 501B of FIG. 5A. Further, the waveform of FIG. 5B does not exhibit period of constant pCO₂. For example, a tangent line 501H of the waveform at point H has an increasing slope in comparison to tangent line 501D of FIG. 5A (which has a slope that is substantially zero). The segment between points I and J on the waveform illustrate the rapid decline in detected CO₂ as the patient enters into the next inspiration.

Exemplary, but non-limiting, features of data 207 that are analyzed in step 424 of process 400 include the slopes of tangent lines 501B, 501C and 501D of points B, C and D, respectively, on the graphical CO₂ waveform of a healthy person as shown in FIG. 5A, and the slopes of tangent lines 501G and 501H of points G and H, respectively, on the graphical waveform of a person with pulmonary obstructive disease, as shown in FIG. 5B. Additional, but non-limiting, data to be analyzed includes the duration of the entire expiratory cycle (e.g., between points A, B, C, D, E and F of the waveform recorded from a healthy person as shown in FIG. 5A and between points A, G, H, I and J of the waveform recorded from a person with pulmonary obstructive disease as shown in FIG. 5B).

In one example of operation, a child carries the portable capnograph device 200, FIG. 2, along with him/her on a camping trip. As instructed by a camp counselor, the child may breathe normally into device 200 such that device 200 performs an analysis of the child's present pulmonary status by comparing the immediate breathing session with personal baseline 210. Device 200 then outputs the determined relative pulmonary status to the camp counselor (or other caregiver) who may be unfamiliar with the child's history. The device may also give a real-time pulmonary status and/or instruction about appropriate decision-making with regard to medication administration and/or the need for more advanced health care.

In another example of operation, a child or adult may use device 200 at approximately the same time daily, indicating on a written or electronic record his or her subjective feelings about respiratory status. On “good” days the device's acquired data would enter the “baseline” database, and on “bad” or “questionable” days the data would be used in comparison with baseline to provide an indication of true pulmonary status. Alternatively, the daily check would be done only against a known baseline approved by a health care provider or against the population norm, in this case providing an early warning of abnormal function that may be unnoticed by the user, and prompting corrective action such as medication dose or health care provider contact. Digital information from the device can be shared with healthcare providers remotely to speed appropriate care and to defer unnecessary face-to-face visits.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present methods and systems, which, as a matter of language, might be said to fall there between.

What is claimed is:

1. An effort-independent, portable, user-operated capnograph device, comprising:
a sensor for sensing concentrations of carbon dioxide (CO₂) exhaled by a user when breathing normally;
a memory for storing data of the sensed concentrations of CO₂ and one or more baseline measurements; and
an analyzer for comparing the stored data with at least one of the baseline measurements to determine the pulmonary status of the user; and
an output device for indicating the pulmonary status to the user.

2. The device of claim 1, wherein at least one of the baseline measurements is a personal baseline uniquely pertaining to the user.

3. The device of claim 1, wherein at least one of the baseline measurements is a population baseline.

4. The device of claim 1, the output device comprising a display for graphically indicating the pulmonary status.

5. The device of claim 4, the display comprising one or more of a liquid crystal display (LCD) and at least one light emitting diode (LED).

6. The device of claim 4, the display showing the pulmonary status using one or more of a graphical waveform, at least one icon, and text.

7. The device of claim 4, the display showing a graph for visually indicating trends in the pulmonary status of the user.

8. The device of claim 1, the output device comprising a speaker for audibly indicating the pulmonary status.

9. The device of claim 8, the speaker outputting the pulmonary status using a synthesized voice.

10. The device of claim 8, the speaker outputting the pulmonary status using a recorded voice.

11. The device of claim 1, further comprising a fingerprint scanner for identifying the user to prevent miss-use.

12. The device of claim 1, further comprising a user interface for receiving identification information from the user, the identification information being used to verify the identity of the user to prevent miss-use.

13. The device of claim 1, further comprising an interface port for connecting to either or both of a computer and a printer.

14. The device of claim 13, the interface port comprising a universal serial bus (USB) port.

15. The device of claim 1, the analyzer mathematically analyzing a graphical waveform representation of the stored data.

16. The device of claim 15, the analyzer comparing features of the graphical waveform against graphical features represented by the baseline measurement.

17. The device of claim 1, the analyzer comparing the stored data with at least one of the baseline measurements in real time.

18. The device of claim 1, the analyzer establishing a new baseline measurement based upon the stored data.

19. A method for evaluating a user's pulmonary status using a portable, user-operated capnograph device, comprising:
storing, within a memory of the capnograph device, at least one baseline measurement;
verifying the identity of a user of the capnograph device to prevent miss-use;
sensing concentrations of carbon dioxide (CO₂) exhaled by the user when breathing normally;
storing, within the memory, data of the sensed concentrations of CO₂;
comparing the stored data with at least one of the baseline measurements to determine the pulmonary status of the user; and
indicating the determined pulmonary status to the user.

20. The method of claim 19, further comprising storing a new baseline measurement within the memory based upon the stored data.

21. The method of claim 19, further comprising providing instruction to the user for operating the capnograph device.

22. The method of claim 21, the instruction comprising verbal instructions.

23. The method of claim 22, the verbal instructions being output using a synthetic voice.

24. The method of claim 22, the verbal instructions being output using a recorded voice.

25. The method of claim 19, further comprising providing instruction to the user based upon the determined pulmonary status.

26. The method of claim 19, wherein at least one of the baseline measurements is a population baseline.

27. The method of claim 19, the step of indicating comprising displaying a graph indicating a trend in the pulmonary status of the user, the trend being based upon the stored data and stored data of a previous session.

28. The method of claim 19, the step of indicating comprising displaying icons to indicate the pulmonary status.

29. The method of claim 19, the step of indicating comprising audibly outputting the pulmonary status using a voice synthesizer.

30. The method of claim 19, the step of indicating comprising audibly outputting the pulmonary status using a recorded voice.

31. The method of claim 19, the step of comparing further comprising graphically analyzing the stored data to identify characteristics for comparison against the baseline measurement.
